(19) United States
(12) Patent Application Publication
(10) Pub. No.: US 2014/0236204 A1
(43) Pub. Date: Aug. 21, 2014

(54) SURGICAL INSTRUMENT

(71) Applicant: LT technologies GmbH & Co. KG, Wurmlingen (DE)

(72) Inventors: Matthias Arit, Neuhausen (DE); Claus Wiest, Wurmlingen (DE)

(73) Assignee: LT technologies GmbH & Co. KG, Wurmlingen (DE)

(21) Appl. No.: 14/181,992

(22) Filed: Feb. 17, 2014

(30) Foreign Application Priority Data
Feb. 18, 2013 (DE) 10 2013 101 594.8

(51) Int. Cl. A61B 17/3205

(52) U.S. Cl. CPC A61B 17/32053
USPC 606/184

(57) ABSTRACT

In a surgical instrument with a shaft part (1), to which a slide (3) is assigned, wherein shaft part (1) and slide (3) are releasably connected to each other, a detent element (14) is intended to protrude from the shaft part (1) and/or from the slide and, as the slide (3) slides along the shaft part (1), the detent element (14) enters a recess (13) of the slide (3) and/or of the shaft part.

### BACKGROUND OF THE INVENTION

The invention relates to a surgical instrument with a shaft part, to which a slide is assigned, wherein shaft part and slide are connected releasably to each other.

Surgical instruments are known, and commercially available, in many shapes and forms. The present invention relates especially, but not exclusively, to sliding shaft instruments, which are also designated as surgical punches. However, the invention can also conceivably be applied to other instruments in which a displaceable element can be changed in position with respect to a relatively fixed element, wherein generally, but again not exclusively, a jaw part is actuated.

A surgical instrument of this kind with scissor grips is known from DE 299 22 271 U1, for example. It is composed of a first grip branch and a second grip branch, wherein the first grip branch is pivotable or expandable relative to the second grip branch and can be held at a distance from each other by means of a spring element. A shaft is connected to the second grip branch. A sliding element that can be coupled to the first grip branch is provided on the shaft, wherein the sliding element, in a position in which the two grip branches are at a maximum distance from each other, can be detached from the shaft and, in the other positions, is axially displaceable on the shaft by pivoting of the first grip branch relative to the second grip branch. Something similar is also known from DE 199 49 422 A1, DE 197 48 369 C2 or also EP 1 363 542 B1. In the latter document, a limit stop for a groove block is also provided, which prevents the groove block from sliding out of a guide groove. This limit stop is oriented obliquely, preferably from below, at an obtuse angle to the guide path.

The object of the present invention is to create a surgical instrument of the abovementioned type which is not only easy to dismantle but also easy to reassemble. Moreover, the concept of the invention should also be able to be applied to previously known instruments.

### SUMMARY OF THE INVENTION

The foregoing object is solved by the fact that a detent element protrudes from the shaft part and/or from the slide and, when the slide is placed onto the shaft part, the detent element enters a recess of the slide and/or of the shaft part.

This arrangement makes assembly of the surgical instrument much easier. Shaft part and slide need only be pressed together with two fingers, after which the slide is moved relative to the shaft part until a control edge of the recess moves across the detent element and the latter enters the recess. For release, the detent element simply has to be pulled out of the recess, as a result of which the slide comes free from the shaft part.

In the context of the invention, it is possible for the detent element to be provided on the shaft part or also on the slide. Its mating piece, i.e. the recess, is then located in or on the respective other part. It is also conceivable to provide a plurality of detent elements, which can be arranged on the shaft part and/or on the slide.

The terms shaft part and slide are to be understood in a broad sense. As has already been mentioned above, the present invention relates especially to a surgical punch, although the inventive concept is also intended to cover other surgical instruments, for example tubular shaft instruments. In these, a pulling or pushing element is displaceable in a corresponding tube in order to actuate a jaw. Here too, the arrangement according to the invention is possible.

Preferably, the detent element should latch automatically into the recess as the control edge crosses it. This is done most simply under the pressure of an energy storage means. The latter, again for reasons of simplicity, is a helical spring.

The whole detent element can be designed as one unit, for example, and can be inserted into a correspondingly formed bore in the shaft part or slide. For simplicity, the detent element is here composed of a pin, which is connected to a button for actuating the pin. The pin is then supported, in the interior of a small housing, against the energy storage means. However, many possibilities are conceivable here and are intended to be covered by the inventive concept.

The slide and shaft part of surgical punches are usually coupled to each other by means of a connection, such that the slide can be displaced relative to the shaft part. Here, groove blocks engage in corresponding undercut grooves such that, during the displacement, the slide cannot come loose from the shaft part. In this case, it may prove advisable to assign the detent element to this undercut groove, i.e. the detent element passes through the undercut groove. For the groove block, an elongate recess is then provided before the undercut groove, into which elongate recess the groove block can be inserted, such that it slides into the undercut groove and beneath the undercuts during the displacement of the slide.

If slide and shaft part are components of a surgical instrument, they are also preferably assigned grip parts. In the present illustrative embodiment, the shaft part merges in one piece into a grip part. This grip part then has, for example in the upper area, an elongate slot through which the grip part for the slide can be inserted. A hinged connection to the slide is located at the end of this grip part, wherein the hinged connection can also be configured such that the slide can be released from the grip part. This makes it much easier to clean the whole instrument.

The concept of the present invention has the further considerable advantage that existing surgical instruments, in particular punches, can be also retrofitted with the arrangement of the detent element and of the recess interacting with the detent element. It is sufficient here for the recess, in particular an oblong hole, to be formed in a corresponding inner face of the slide, and for the detent element with the pin to be arranged, lying opposite the recess, in the shaft part. Only one bore is necessary here. A reverse arrangement is of course also possible, in which the recess is introduced into an upper surface of the shaft part and the detent element is introduced into a bore in the slide.

A reworking of the groove block and of the undercut groove may then also be necessary in order to adapt them to the desired functions.

### BRIEF DESCRIPTION OF THE DRAWINGS

Further advantages, features and details of the invention will become clear from the following description of preferred illustrative embodiments and by reference to the drawing, in which:

FIG. 1 shows a plan view of a surgical instrument according to the invention;

FIG. 2 shows a partial plan view of a shaft part of the surgical instrument;

FIG. 3 shows a partial bottom view of a slide part of the surgical instrument according to FIG. 1;

FIG. 4 shows a partial side view of a detail of slide and shaft part;

FIG. 5 shows an enlarged cross-sectional view of a detail x from the shaft part according to FIG. 4.

### DETAILED DESCRIPTION

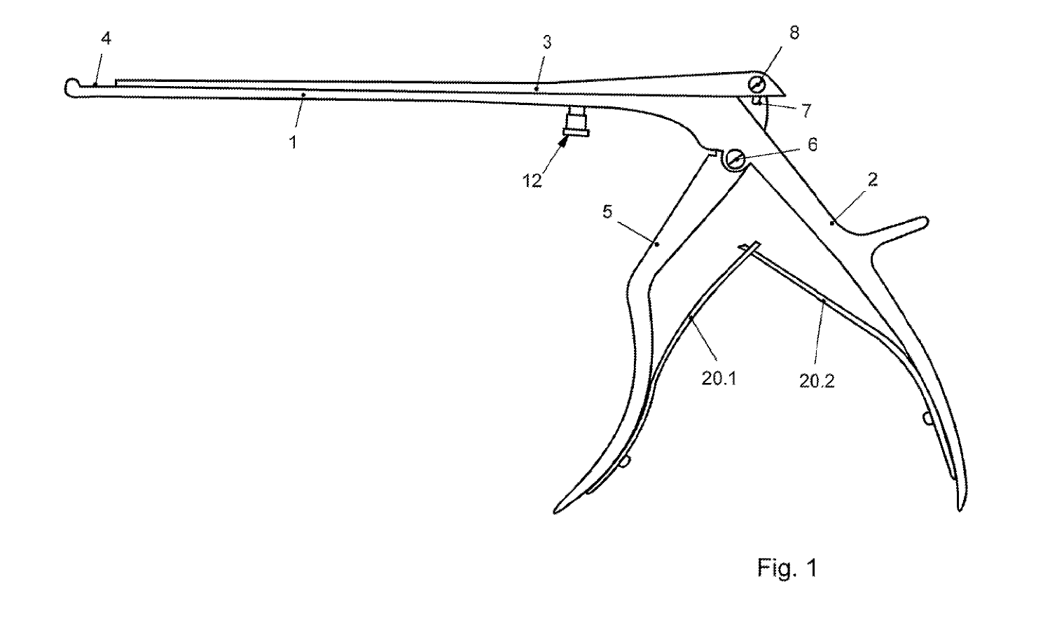

In accordance with FIG. 1, a surgical instrument P according to the invention has a shaft part 1 which, in the illustrative embodiment shown, merges in one piece into a grip part 2. A slide 3 sits on this shaft part 1 and is movable along the shaft part 1. During this movement, a jaw 4, which is formed toward the end between shaft part 1 and slide 3, is opened or closed. In so doing, a piece of tissue, for example, can be punched out.

The movement of the slide 3 is effected from a further grip part 5, which passes through the other grip part 2 and is connected to the latter via a hinge 6. An oblong hole 7 is formed in this grip part 5, at the end thereof protruding from the grip part 2, into which oblong hole 7 a transverse bolt 8 is fitted, such that a hinged connection to the slide 3 is formed here.

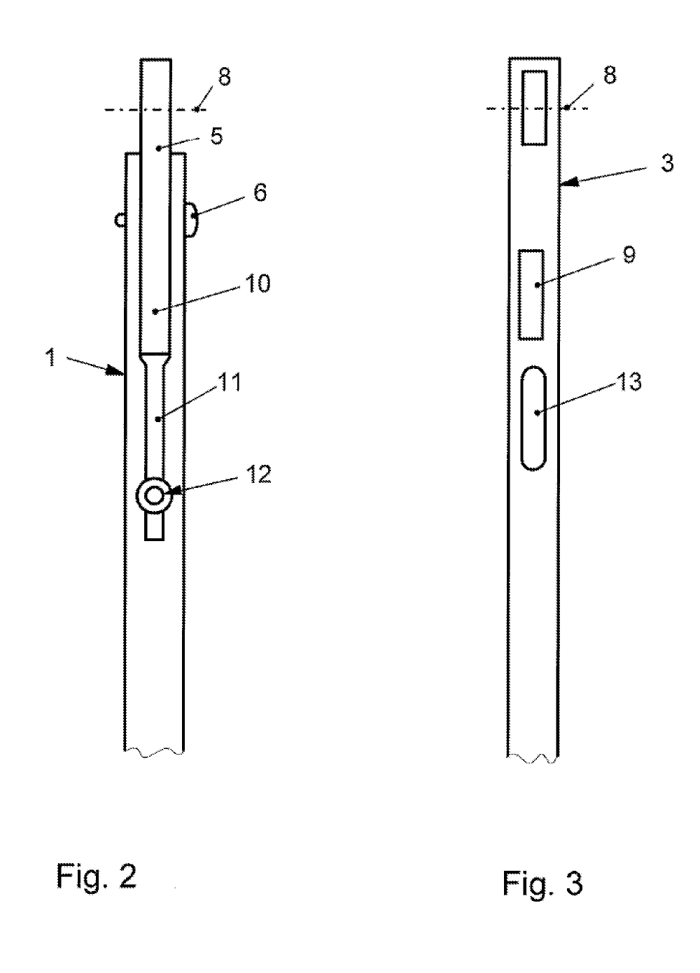

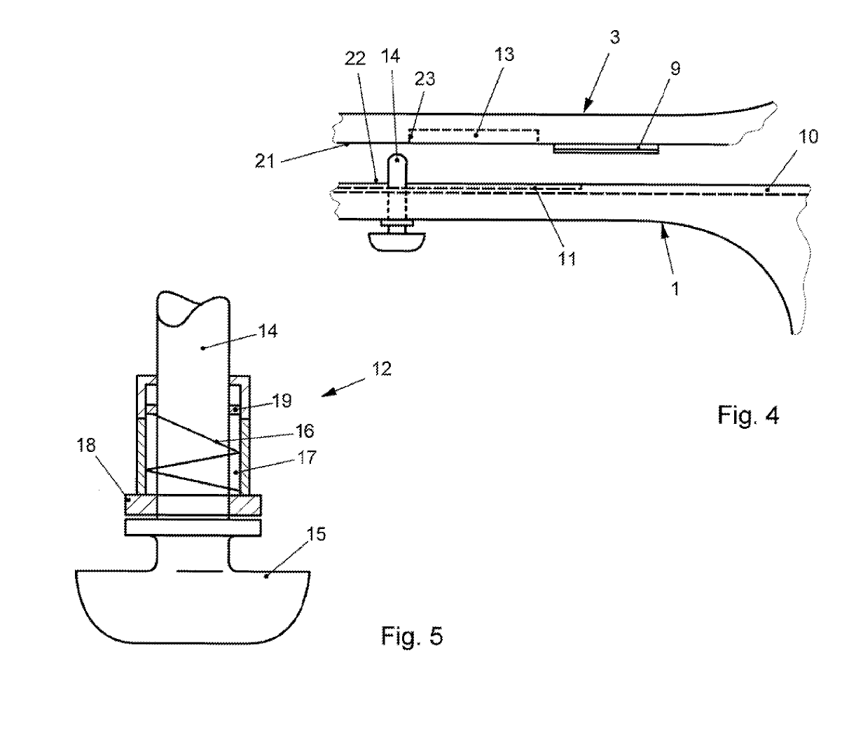

The slide 3 is connected releasably to the shaft part 1. For this purpose, a groove block 9 protrudes downward from the slide 3 as shown in FIG. 4, which groove block 9 can be inserted into a recess 10 (see also FIGS. 2 and 3) in the shaft part 1 and, after a sliding travel in the direction of the jaw 4, enters an undercut groove 11.

To prevent the groove block 9 from sliding rearward out of the undercut groove 11 away from the jaw, a detent means 12 according to the invention is provided. This detent means interacts with an oblong hole 13 in the slide 3. In the illustrative embodiment shown, the detent means 12 passes through the shaft part 1 in the area of the undercut groove 11, although it could also be arranged at another location. It has a pin 14, which can be actuated by a button 15. The actuation takes place counter to a helical spring 16, which is located in a bore 17 in the shaft part 1. The helical spring 16 is supported, at one end, against a wall 18 of the shaft part 1 or against a separately inserted plate and, at the other end, against a retaining ring 19 fitted on the pin 14.

The function of the present invention is as follows:

In an initial position, the two grips parts 2 and 5 are connected to each other via the hinge 6, which is formed by a screw bolt, for example. They are mutually supported by two leaf springs 20.1 and 20.2, such that the jaw 4 is held in the open position.

As is shown in FIG. 4, shaft part 1 and slide 3 are not in the latching position. In order now to connect the slide 3 to the shaft part 1, this slide 3 is moved toward the shaft part 1, wherein the pin 14 protruding from the shaft part 1 is forced away by an inner face 21 of the slide 3. This takes place counter to the force of the helical spring 16. At the same time, the groove block 9 enters the recess 10 behind the undercut groove 11. In this position, the inner face 21 lies flat on an upper face 22 of the shaft part 1.

By means of the grip part 5, the slide 3 is now moved in the direction of the jaw 4, whereupon the groove block 9 enters the undercut groove 11 and engages below the corresponding undercuts. The slide 3 is thereby fixed with respect to the shaft part 1.

After a further distance is traveled, the oblong hole 13 in the slide 3 moves across the pin 14, such that the latter can enter the oblong hole 13, behind a control edge 23 of said oblong hole 13. The helical spring 16 thus relaxes. By bearing on the pin 14, this control edge 23 now has the effect of limiting the spreading action of the leaf springs 20.1 and 20.2 against the grip parts 2 and 5. This avoids a situation in which the groove block 9 moves too far toward the rear and in so doing inadvertently escapes from the undercut groove 11.

To dismantle the surgical instrument according to the invention, the pin 14 is simply moved out from the latching position in the oblong hole 13 by pulling the button 15, such that the control edge 23 can now move across the pin 14. After a further distance is traveled by the slide 3 with respect to the shaft part 1, the groove block 9 also disengages from the undercut groove 11. The slide 3 can now be lifted away from the shaft part 1. For example, if the oblong hole 7 is open toward the top, the slide 3 can be detached from the grip part 5 even without releasing the transverse bolt 8.

1. Surgical instrument comprising a shaft part, a slide movably located on the shaft part, wherein shaft part and slide are releasably connected to each other, a detent element protrudes from the shaft part and/or from the slide and, as the slide slides along the shaft part, the detent element enters a recess of the slide and/or of the shaft part.

2. Instrument according to claim 1, wherein the detent element is held in a latching position under the pressure of an energy storage means.

3. Instrument according to claim 2, wherein the energy storage means is a helical spring.

4. Instrument according to claim 3, wherein the energy storage means is fixed in a bore in the shaft part and/or of the slide.

5. Instrument according to claim 1, wherein the detent element has a pin, which is connected to a button for actuating the pin.

6. Instrument according to claim 1, wherein the detent element passes through an undercut groove in the shaft part.

7. Instrument according to claim 6, wherein the undercut groove in the shaft part is adjoined by an elongate recess.

8. Instrument according to claim 1, wherein a recess in the slide comprises an oblong hole which receives the detent element.

9. Instrument according to claim 7, wherein the recess in the slide is provided lying approximately opposite the undercut groove.

10. Instrument according to claim 1, wherein the recess is located in the slide and is adjoined by a groove block.

11. Instrument according to claim 10, wherein the groove block is provided in the shaft part approximately opposite the recess.

12. Instrument according to claim 1, wherein both the shaft part and the slide are adjoined by separate grip parts.

13. Instrument according to claim 12, wherein the separate grip parts are connected to each other in a hinged manner.

14. Instrument according to claim 12, wherein the grip part assigned to the slide is connected to the slide in a hinged, releasable manner.
